(19) United States
(12) Patent Application Publication
(10) Pub. No.: US 2007/0161868 A1
(43) Pub. Date: Jul. 12, 2007

(54) Method and system for determining whether additional laboratory tests will yield values beyond a threshold level

(75) Inventor: Martin Menzo Root, Pittsboro, NC (US)

Correspondence Address: DANIELS DANIELS & VERDONIK, P.A., SUITE 200 GENERATION PLAZA, 1822 N.C. HIGHWAY 54 EAST, DURHAM, NC, 27713, US

(73) Assignee: BioSignia, Inc., Durham, NC (US)

(21) Appl. No.: 11/329,214

(22) Filed: Jan. 10, 2006

(51) Int. Cl. A61B 5/00

(52) U.S. Cl. 600/300; 128/920

(57) ABSTRACT

A method and system is provided which takes into account actual human data previously collected. The human data is used to predict the probability of a test conducted on a patient later, yielding a result above a threshold level which would be informative to a doctor treating a patient. The test which is conducted is for detecting levels of C-reactive protein, lipoprotein(a) or homocysteine in a patient. Patient data is collected and input into a multivariate function which then yields a percentage probability that test results on such a patient will be above a predetermined level.

### BACKGROUND OF THE INVENTION

1. FIELD OF THE INVENTION

This invention relates to a method and system for determining whether ordering of more expensive tests for a patient is likely to yield useful results. More specifically, the invention relates to a method and system for determining whether ordering an additional laboratory test for measuring levels of C-reactive protein (CRP), lipoprotein (a) (LPA) or homocysteine (HCY) in a patient is likely to yield results above a predetermined threshold level which may be indicative of increased risk of heart disease in the patient.

2. DISCUSSION OF THE RELATED ART

Currently, doctors are often faced with the choice of whether to order more expensive tests for a patient depending on the results obtained from an initial battery of tests, which while providing useful results, may be inconclusive. Doctors have to balance the costs, inconvenience and pain of the tests against the benefit of additional information which such a test result may yield. One particular marker is C-reactive protein which is an indicator of systemic inflammation in a patient. Current research has shown that C-reactive protein (CRP), if the results of a test are above a certain level, may be predictive of heart disease when combined with other factors. Other similar tests for heart disease include tests for lipoprotein(a) (LPA) and homocysteine (HCY) levels.

While these markers are informative, obtaining such marker values can also be very expensive. Moreover, to some extent the markers by themselves only provide limited predictability with respect to certain specific conditions such as heart disease. For example, people with diabetes and people who are obese generally have high CRP levels. Since both diabetes and obesity are already risk factors for heart disease, the marker by itself is not particularly useful. A doctor treating a patient is thus often left with a difficult decision as to whether to have a patient undergo a test for the particular marker, in this case, CRP, because the results of the tests may not be of much help.

Current approaches to deciding whether to test for a marker such as CRP, LPA or HCY are usually hit-or-miss. Typically, the decision is based on the doctor's training, experience, intuition and past prejudices. Some approaches have involved a set of rules. In the case of multiple sclerosis, for example, the doctor may review a few simple questions such as family history of multiple sclerosis (MS), telltale symptoms, age, rate of onset, and may be asked to count up the number of positive results. Accordingly, once a certain threshold is reached, a test is recommended.

Reflex testing is one way these decisions are currently made. In a common reflex testing situation, a blood sample is sent to a lab for a quick screening test of a variety of infectious diseases that the patient may have. When one of these tests comes up positive, the sample is automatically (“reflexively”) resubmitted for a more expensive and specific test for that one microbe.

In the case of a patient with a risk for heart disease, a doctor may determine the patient has a high risk based on cheap and easy tests such as cholesterol and blood pressure. The doctor may then consider more expensive and newer tests based on the patient's currently known high risk level.

In accordance with the invention, the haphazard and hit-or-miss results of past techniques are avoided. A more systematic and scientific means of deciding on future tests is here described.

### BRIEF SUMMARY OF THE INVENTION

In accordance with the invention, there is provided a method and system which is based on actual human data used to predict the probability of a test conducted later yielding a result above a threshold level which would be informative to a doctor treating a patient.

In accordance with one specific implementation, the invention is directed to a system and method for determining whether ordering an additional laboratory test measuring levels of at least one of C-reactive protein (CRP), lipoprotein(a) (LPA), and homocysteine (HCY), in a patient is likely to yield useful results.

The method and system implements a multivariate function which yields a percentage result of the number of people in a given population, with like acquired human data, who would be likely to yield results above a certain threshold of the additional test if such test were performed. Similar multivariate functions can be applied in either one of testing for CRP, LPA or HCY. The invention also includes a computer program product implementing the multivariate function.

In accordance with one method, values are determined for a patient, including at least the sex of the patient, age of the patient, the blood glucose level of the patient, the serum albumin level of the patient, the coronary heart disease state of the patient and the body mass index. The values are inserted into a function to yield a result. A predetermined threshold was previously established for the CPR test. The result of the function is the probability that an additional laboratory test for measuring C-reactive protein in a patient is likely to yield a useful result above the threshold for that patient.

While a first embodiment uses prediction of high CRP levels, the invention can also be implemented in a predictive manner for results of further testing for lipoprotein(a) and/or homocysteine.

In the lipoprotein(a) case, the values determined include at a minimum the age of the patient and whether the patient is of African American ethnic origin. In a more complete/complex model, values for TCHOL (serum total cholesterol in mg/dL), LDL (low density lipoprotein) cholesterol and HDL cholesterol are also factored into the calculation. Also as in the case with CRP and HCY, these values are factored into a multivariate function which provides a probability that a percentage of patients with like values inserted into the respective equation will yield useful diagnostic results above a predetermined threshold if additional tests are ordered.

For HCY, the values determined include at a minimum: the age and the sex of the patient, particularly whether the patient is female. In a more complete/complex model, other values include whether the patient is Hispanic, a smoker and the patient's HDL (high density lipoprotein) cholesterol level. These are factored into a multivariate function as is done in the case with CRP.

For CRP, if the result of the calculation yields a value of 10%, this means that of the total population of patients with similar values, then about 10% of the population will have a CRP value greater than 10 mg/L, which may serve as a cutoff value for a clinician deciding to run a test for CRP levels. In a more common practice with a slight difference for a CRP>3 mg/L, if the percent result is at least 10%, this indicates that about 10% of the population with similar values will have a CRP>3 mg/L, placing the particular patient in that group, and establishing a second more preferred threshold. The decision about whether to actually order the CPR test for a specific patient is left to the discretion of the doctor. The function only estimates for a particular patient the likelihood that an ordered test will have a result over the threshold of 3 or 10 mg/L.

In the case of LPA, if the result of the function is 10% or greater, this means that at least 10% of the patients with similar values will have an LPA level of greater than 30 mg/dL.

In the case of HCY, if the result of the function is 10% or greater, this means that at least 10% of the patients with similar values will have an HCY level greater than 15 μmol/L.

Depending on the result of each equation, a skilled doctor can make a decision on whether to order an additional test as will become clearer from the remainder of the description herein.

In another aspect, the invention involves a system for conducting the afore-described method. The system includes means for inputting at least one of patient test data and doctor data into a database. A database serves for storing input data relating to individual patients, including at least one of patient test data and doctor data. A program product on the system serves for receiving patient data, including at least one of the patient test data and doctor data for computing the probability that additional laboratory tests for at least one of CRP, LPA and HCY will yield useful results. The program product does the computation through at least one multivariate function. Means is provided to provide the results of the computation to a clinician.

### BRIEF DESCRIPTION OF THE SEVERAL VIEWS OF THE DRAWING

Having thus briefly described the invention, the same will become better understood from the following detailed discussion presented herein with reference to the accompanying drawing wherein:

FIG. 1 is a block diagram illustrating how test data is input into a database and then run through program modules to result in a report indicating the probability that additional testing will yield useful results;

FIG. 2 illustrates a typical computer system on which the method of the invention can be implemented to result in a report indicating whether additional testing will yield useful results;

FIG. 3 is a block diagram illustrating typical components of such a computer;

FIG. 4 illustrates a typical report based on test data operated on in accordance with the method of the invention; and

FIG. 5 is a block diagram illustrating the physical steps involved in a typical scenario where the patient and physician interact to obtain clinical data and samples which are then operated in accordance with the method of the invention to result in an output report.

### DETAILED DESCRIPTION OF THE INVENTION

As previously briefly explained, medical testing has become increasingly expensive, such that it becomes important to reduce the number of tests conducted on a patient, if additional testing is unlikely to yield useful results for diagnoses and treatment purposes. In the area of heart disease, it is known that if tests for C-reactive protein (CRP), lipoprotein(a) (LPA) and homocysteine (HCY) are above certain values, they are predictive of coronary heart disease coronary heart disease (CHD). However, if those tests fall below a certain value, the results of the tests are essentially useless to a physician because they give no additional information concerning a person's risk of CHD.

While these are informative markers concerning a person's health, the tests to obtain the values are very expensive and thus, there is a hesitancy to order such tests unless the physician in his or her subjective judgment believes that the tests are likely to yield useful results.

In accordance with the invention described herein, it has been discovered that the results of tests conducted for the afore-mentioned markers are, to a limited extent, predictable.

In implementing the invention, the National Health and Nutrition Examination Survey 1999-2000 (NH2K) was used. This survey is a comprehensive survey of several thousand American adults that obtained information among other things, blood tests and other disease related risk factors. The results of the survey are used in particular, in accordance with the invention, with respect to determining the likelihood that a test for CRP, LPA or HCY would result in values above a certain threshold. CRP, as is well known, is a marker for systemic inflammation and at high levels is indicative of acute insult, infection or injury. At lower but still higher than normal levels it is a risk factor indicating possible coronary heart disease or cardiovascular disease. LPA and HCY are also indicators of possible coronary heart disease or cardiovascular disease.

Thus, in one aspect as applied to CRP, there is provided a method of determining whether ordering an additional laboratory test for measuring levels of C-reactive protein in a patient is likely to yield results above a predetermined threshold. The method involves determining values for a patient including at least the sex, age, blood glucose level, the serum albumin level, the coronary heart disease state and the body mass index. These values are inserted into a predetermined multivariate mathematical function which will result in a value which is the probability of a high value for C-reactive protein that is likely to result for the patient if a test for C-reactive protein is conducted on the patient.

The values are introduced into a multivariate function to yield a probability of a high value.

Thus, if the result of running the function yields 13%, this means that 13% of the population with similar input values will have a CRP level of greater than the determined threshold.

In a preferred aspect, the values are inserted into the function.
% probability=1/(1+exp(−(−5.669+0.776*FEMALE+0.1028*BMI)))
where:

% probability=the probability that a patient has a CRP>10 mg/L

FEMALE=1 for a female patient and=0 for a male patient

BMI=a patient's body mass index in units of kg/m², where kg is the body weight in kilograms and m is the patient's height in meters squared.

As an alternative, it is possible that a CRP of 3 may also be a cutoff and those who are predicted to have values above that may be good candidates for additional testing. In that case, the function for the cutoff value explained hereafter is as follows:
% probability=1/(1+exp(−(−4.681+0.773*FEMALE+0.133*BMI)))

Thus, if the result of running the function yields about 13%, this means that about 13% of the population with similar values will have a CRP level of greater than 3.

These and other models and functions described herein were invented through the use of the National Health and Nutrition Examination Survey (NHANES) from 1990-2000 (NH2K). They were calculated by weighted multivariate logistic analysis. They were weighted according to the weighting scheme of NH2K to be representative of all adult Americans.

In a more detailed representation of multivariate logistic analysis for a CRP of greater than 10 mg/dL, the following function may be used:
% probability=1/(1+exp(−(−0.040+1.154*FEMALE+0.0174*AGE−0.0120*FEMALE*AGE+0.496*CHD+0.0861*BMI−1.377*ALB+0.00164*GLUCOSE)))
where:

AGE=age in years;

CHD=a doctor's diagnosis of coronary heart disease=1 else=0;

ALB=serum albumin in g/L; and

GLUCOSE=serum glucose in mg/dL.

This function is used for a cutoff value for CRP of greater than 10 mg/L, thus providing a more accurate calculation of the first function above which uses only FEMALE and BMI as input variables.

In a more detailed representation for a CRP>3 mg/dL, the following function may be used:
% probability=1/(1+exp(−(0.13427+(0.81917*FEMALE)+(0.01489*AGE)−(0.00571*FEMALE*AGE)+0.21976*CHD+(0.10436*BMI)−(1.49181*ALB)+(0.43003*LOGTRIG))))
where:

LOGTRIG=natural logarithm of serum triglycerides in mg/dL.

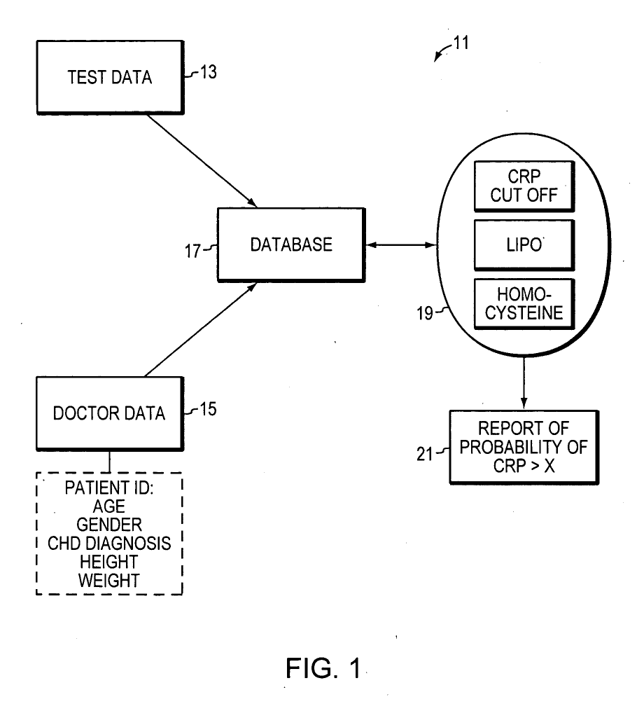

The method of the invention is better illustrated in FIG. 1, wherein a block diagram 11 illustrates that patient test data 13 such as coming from conducting a blood test is added to a database 17. The patient data for the blood test can be as previously discussed, and includes blood glucose level and the albumin level of the patient. Doctor data 15 can also be inserted into the database and can include such items as the patient ID, the age, the gender, the coronary heart disease (CHD) state, height, weight and body mass index (BMI) of the patient. The data collected is then run through a program in which it is inserted into the afore-mentioned function. The program modules 19 can be a CRP cutoff module, but although CRP is indicated as a specific implementation herein, the data could also be input into a lipoprotein or homocysteine module to then yield a report 21 of the probability of the CRP if tested for the later date resulting above a certain predetermined value. Similarly, while CRP is indicated, the report can also indicate whether the lipoprotein(a) value or homocysteine value will result above a predetermined value as a result of testing.

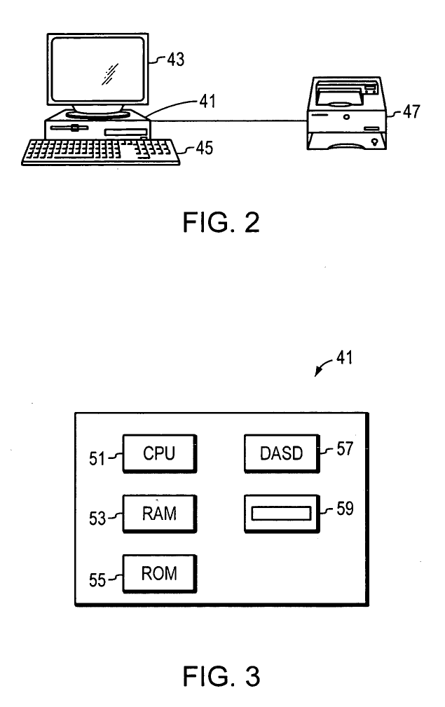

FIG. 2 illustrates a typical system upon which the method of the invention can be implemented. Such a system can involve a computer 41 having a display 43 and means for inputting at least one of patient test data and doctor data into the database 17 such as through keyboard 45. The computer 41 can have connected thereto alternative means for inputting such as a network cable (not shown) for inputting the results, for example, blood tests, that might be conducted in a laboratory such as through communications through a network. Similarly, as already noted, patient data can be input through means for inputting such as through a keyboard 45 and once the modules are run, the results can be output through means for providing results such as through a printer 47 or display 43.

FIG. 3 shows in greater detail the makeup of a typical computer 41 which would include a CPU 51 with RAM 53, ROM 55, a direct access storage device 57, for example, for storing the program modules therein as well as the data to be input into the afore-described function. Such a device 57 can be a hard drive, for example. An external drive 59 can also be provided for inputting additional data as necessary.

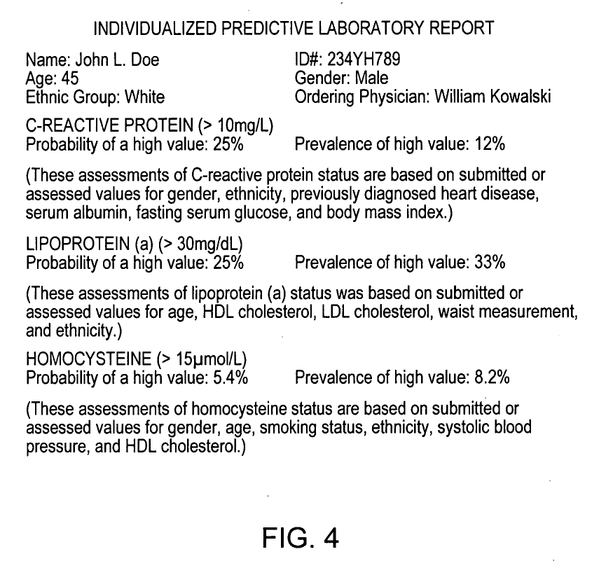

FIG. 4 illustrates a sample report indicating how the results will differ depending on data input for the patient. The sample report of FIG. 4 is designed for providing physicians information that would help in making a decision to order or not to order additional laboratory tests. The report provides a balance between providing information and trying to force the physician's hand.

The report of FIG. 4 is an individualized report based on specific information submitted on an interview sheet. It makes use of interview data, clinical data and laboratory test data. The calculations are based on NHANES III and NHANES 2000 data, and on statistical models of those data.

In the report, for each biomarker, each section starts with the name of the potential test biomarker and the cutoff value being used in that analysis. In the first section, the concern is the probability of a patient having a high CRP if the test were ordered. Based on the risk factors, the patient's probability of having a CRP>10 mg/L is indicated at 25%. Considering the prevalence for the population is 12%, this corresponds to setting the threshold at which tests were ordered at 10% or greater for an indicated CRP level if greater than 10 mg/L.

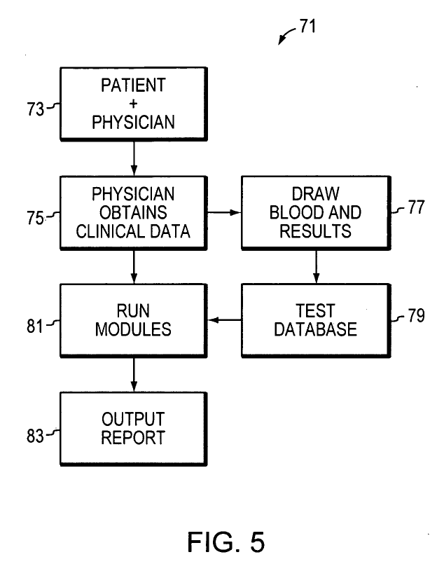

FIG. 5 illustrates in greater detail a block diagram showing the physical steps involved in obtaining the data and running it through the modules to obtain an output report in accordance with the method of the invention. In the block diagram 71 of FIG. 5, the patient and physician initially meet 73. At steps 75, the physician obtains clinical data, for example, by physical examination of the patient, with the data being of the type previously discussed including age, gender, etc. The physician or an assistant then draws blood 77 and obtains the results of blood tests run on the blood which is then input into a test database 79. The clinical data obtained at steps 75 is then input along with the data from the blood tests into the program and the modules depending on whether it is a CRP, lipoprotein or homocysteine module run to result in an output report at step 83.

The aforementioned method and system can, as previously discussed, also implemented with LPA and HCY.

In the case of LPA, a typical minimum representative multivariate function is:
% probability=1/(1+exp(−(−1.424+0.0000744*AGE²+1.615*BLACK))),
where:

% probability=the probability that a patient has a LPA>30 mg/dL;

BLACK=African American ethnic origin=1 else=0.

A more complete function for LPA is:
% probability=1/(1+exp(−(−2.480+0.0000369*AGE²+1.590*BLACK−0.0107*TCHOL+0.0161*HDL+0.0198*LDL))),
where:

TCHOL=serum total cholesterol in mg/dL;

LDL=low density lipoprotein cholesterol in mg/dL.

With respect to HCY, a typical representative multivariate function is:
% probability=1/(1+exp(−(−1.799-0.685*FEMALE−0.0491*AGE+0.00076*AGE²)))
where:

% probability=the probability that a patient has a HCY>15 μmol/L.

A more complete function for HCY is:
% probability=1/(1+exp(−(−1.633-0.574*FEMALE-0.0579*AGE+0.00087*AGE²−0.341*HISP+0.623*SMOKE−0.00622*HDL))),
where:

HISP=Hispanic ethnic origin=1 else=0;

HDL=high density lipoprotein cholesterol in mg/dL;

SMOKE=patient is smoker=1 else=0.

### EXAMPLES

Having given representative implementations of multivariate functions based on NHANES data, the following are representative examples for two people using values for the three markers CRP, LPA and HCY. Fred is the high risk patient and Mary is a low risk patient.

### Example I

| Patient: | Fred Jones |
|---|---|
| Age | 65 |
| Ethnicity | Black |
| Previous CHD | yes |
| Smoking habit | current |
| BMI | 31 kg/m² |
| Albumin | 3.5 g/L |
| Glucose | 107 mg/dL |
| Total CHOL | 190 mg/dL |
| HDL CHOL | 35 mg/dL |
| LDL CHOL | 140 mg/dL |
| CRP >10 mg/L | prob = 41% |
| HCY >15 μmol/L | prob = 21% |
| LPA >30 mg/dL | prob = 64% |

### Example II

| Patient: | Mary Smith |
|---|---|
| Age | 31 |
| Ethnicity | White |
| Previous CHD | no |
| Smoking habit | none |
| BMI | 22 kg/m² |
| Albumin | 4.7 g/L |
| Glucose | 72 mg/dL |
| Total CHOL | 150 mg/dL |
| HDL CHOL | 62 mg/dL |
| LDL CHOL | 71 mg/dL |
| CRP >10 mg/L | prob = 4.0% |
| HCY > μmol/L | prob = 2.8% |
| LPA >30 mg/dL | prob = 16% |

While specific constants and weighting have been indicated for the various functions described herein, the invention is not limited to those specific values. It will be appreciated by those of ordinary skill that the weights and constants can be adjusted, depending on the variables with which employed, to achieve results as described herein.

With respect to the results of the functions, it is important to appreciate that it is the physician or combination of physician and insurance carrier that determines what result is significant enough to order additional tests. For example, a physician may decide that a test result indicating that a patient has a 10% or higher probability of having a CRP level greater than 10 mg/L is sufficient to warrant additional testing, but another physician may decide the threshold should be 20% or higher. Yet still another physician may decide to run the function for a CRP>3 mg/L, and may decide for that function that a result of 30% or higher warrants an additional test. Ultimately, the insurance company may be the final decision maker setting the threshold above which, if additional testing is done, they will cover the costs of the additional test.

It is important to appreciate that the invention provides tools to facilitate physician decisions. It is the physician who practices medicine and makes final decisions. The physician may have reasons beyond factors considered by the various implementations of a multivariate function of the invention for ordering or not ordering a test. For example, the function may yield a high percentage probability of a CRP above a threshold level. However, the physician knows that the patient was recently immunized for a certain disease, and that such immunization results in skyrocketing CRP levels in the days after an immunization having nothing to do with CHD risk. The physician would receive spurious results if a CRP test were ordered at that time.

Having thus briefly described the invention, the same will become better known from the appended claims in which it is set forth in a non-limiting manner.

What is claimed is:

1. A method of determining whether ordering an additional laboratory test for measuring levels of C-reactive protein in a patient is likely to yield results above a predetermined threshold, comprising:
determining values for a patient including at least the sex of the patient and the patient's body mass index;
entering said values into a multivariate function having predetermined weighting values; and
computing the results of the function into said values to result in an output indicative of whether or not said additional test for C-reactive protein is likely to yield useful results.

2. The method of claim 1, wherein said function is:
% probability=1/(1+exp(−(−5.669+(0.776*FEMALE)+(0.1028*BMI)))),
where:
% probability=the probability that a patient has a CRP>10 mg/L;
FEMALE=1 for a female patient and=0 for a male patient; and
BMI=a patient's body mass index in units of kg/m², where kg is the body weight in kilograms and m² is the patient's height in meters squared.

3. The method of claim 1, wherein said function is:
% probability=1/(1+exp(−(−0.040+(1.154*FEMALE)+(0.0174*AGE)−(0.0120*FEMALE*AGE)+(0.496*CHD)+(0.0861*BMI)−(1.377*ALB)+(0.00164*GLUCOSE)))),
where:
% probability=the probability that a patient has a CRP>10 mg/L;
FEMALE=1 for a female patient and=0 for a male patient;
BMI=a patient's body mass index in units of kg/m², where kg is the body weight in kilograms and m² is the patient's height in meters squared;
AGE=age in years;
CHD=a doctor's diagnosis of prior coronary heart disease=1, else=0;
ALB=serum albumin in g/L; and
GLUCOSE=serum glucose in mg/dL.

4. The method of claim 1, wherein said function is:
% probability=1/(1+exp(−(−4.681+(0.773*FEMALE)+(0.133*BMI)))),
where:
% probability=the probability that a patient has a CRP>3 mg/L;
FEMALE=1 for a female patient and=0 for a male patient; and
BMI=a patient's body mass index in units of kg/m², where kg is the body weight in kilograms and m2 is the patient's height in meters squared.

5. The method of claim 1, wherein said function is:
% probability=1/(1+exp(−(0.13427+(0.81917*FEMALE)+(0.01489*AGE)−(0.00571*FEMALE*AGE)+0.21976*CHD+(0.10436*BMI)−(1.49181*ALB)+(0.43003*LOGTRIG)))),
where:
% probability=the probability that a patient has a CRP>3 mg/L;
FEMALE=1 for a female patient and=0 for a male patient;
BMI=a patient's body mass index in units of kg/m², where kg is the body weight in kilograms and m² is the patient's height in meters squared;
AGE=age in years;
CHD=a doctor's diagnosis of prior coronary heart disease=1, else=0;
ALB=serum albumin in g/L; and
LOGTRIG=natural logarithm of serum triglycerides in mg/dL.

6. The method of claim 1, wherein said function is implemented on a computing device as part of a computer program product, and said values for a patient are obtained through at least one of physical examination and medical testing, and the values are then input into the computing device to be operated on by said function, the computer program is then run and the probability is output as part of the report.

7. A method of determining whether ordering an additional laboratory test for measuring lipoprotein(a) (LPA) in a patient is likely to yield results above a predetermined threshold, comprising:
determining values for a patient including at least the age of a patient and whether the patient is of African American ethnic origin;
entering said values into a multivariate function having predetermined weighting values; and
computing the results of the function with said values to result in an output indicative of whether or not said additional test for LPA is likely to yield useful results.

8. The method of claim 7, wherein said function is:
% probability=1/(1+exp(−(−1.424+(0.0000744*AGE²)+(1.615*BLACK)))),
where:
% probability=the probability that a patient has a LPA >30 mg/dL;
AGE=age of the patient; and
BLACK=African American ethnic origin=1, else=0.

9. The method of claim 7, wherein said function is:
% probability=1/(1+exp(−(−2.480+(0.0000369*AGE²)+(1.590*BLACK)−0.0107*TCHOL)+(0.0161*HDL)+(0.0198*LDL)))),
where:
% probability=the probability that a patient has a LPA>30 mg/dL;
AGE=age of the patient;
BLACK=African American ethnic origin=1, else=0;
TCHOL=serum total cholesterol in mg/dL;
HDL=high density lipoprotein cholesterol in mg/dL; and
LDL=low density lipoprotein cholesterol in mg/dL.

10. The method of claim 7, wherein said function is implemented on a computing device as part of a computer program product, and said values for a patient are obtained through at least one of physical examination and medical testing, and the values are then input into the computing device to be operated on by said function, the computer program is then run and the probability is output as part of the report.

11. A method of determining whether ordering an additional laboratory test for measuring Homocysteine (HCY) is likely to yield results above a predetermined threshold, comprising:
determining values for a patient including at least the sex of a patient and the age of the patient;
entering said values into a multivariate function having predetermined weighting values; and
computing the results of the function with said values to result in an output indicative of whether or not said additional test for HCY is likely to yield useful results.

12. The method of claim 10, wherein said function is:
% probability=1/(1+exp(−(−1.799−(0.685*FEMALE)−(0.0491*AGE)+(0.00076*AGE²)))),
where:
% probability=the probability that a patient has a HCY>15 μmol/L;
AGE=age in years; and
FEMALE=1 for a female patient and =0 for a male patient.

13. The method of claim 10, wherein said function is:
% probability=1/(1+exp(−(−1.633−(0.574*FEMALE)-(0.0579*AGE)+(0.00087*(AGE²)−(0.341*HISP)+(0.623*SMOKE)−(0.00622*HDL)))),
where:
% probability=the probability that a patient has a HCY>15 μmol/L;
AGE=age in years;
FEMALE=1 for a female patient and =0 for a male patient;
HISP=Hispanic ethnic origin=1, else=0;
HDL=high density lipoprotein cholesterol in mg/dL; and
SMOKE=patient is smoker=1, else=0.

14. The method of claim 10, wherein said function is implemented on a computing device as part of a computer program product, and said values for a patient are obtained through at least one of physical examination and medical testing, and the values are then input into the computing device to be operated on by said function, the computer program is then run and the probability is output as part of the report.

15. A system for determining whether ordering an additional laboratory test for measuring at least one of the C-reactive protein (CRP), lipoprotein (a) (LPA) and homosysteine (HCY) in a patient is likely to yield results above a predetermined threshold, comprising:
means for inputting at least one of patient test data and doctor data into a database;
a database for storing input data relating to individual patients, including at least one of patient test data and doctor data;
a program product for receiving patient data, including at least one of the patient test data and doctor data for computing the probability that additional laboratory tests for a specific patients will yield useful results, said computing being done through at least one multivariate function for computing whether additional tests for at least one of CRP, LPA, and HCY will yield useful results; and
means for providing said results of said computation to a clinician.

16. The system of claim 15, wherein said multivariate function of said program product is:
% probability=1/(1+exp(−(−5.669+(0.776*FEMALE)+(0.1028*BMI)))),
where:
% probability=the probability that a patient has a CRP>10 mg/L;
FEMALE=1 for a female patient and=0 for a male patient; and
BMI=a patient's body mass index in units of kg/m², where kg is the body weight in kilograms and m² is the patient's height in meters squared.

17. The system of claim 15, wherein said multivariate function of said program product is:
% probability=1/(1+exp(−(−0.040+(1.154*FEMALE)+(0.0174*AGE)−(0.0120*FEMALE*AGE) +(0.496*CHD)+(0.0861*BMI)−(1.377*ALB)+(0.00164*GLUCOSE)))),
where:
% probability=the probability that a patient has a CRP>10 mg/L;
FEMALE=1 for a female patient and=0 for a male patient;
BMI=a patient's body mass index in units of kg/m², where kg is the body weight in kilograms and m is the patient's height in meters squared;
AGE=age in years;
CHD=a doctor's diagnosis of prior coronary heart disease=1, else=0;
ALB=serum albumin in g/L; and
GLUCOSE=serum glucose in mg/dL.

18. The system of claim 15, wherein said multivariate function of said program product is:
% probability=1/(1+exp(−(−4.681+(0.773*FEMALE)+(0.133*BMI)),
where:
% probability=the probability that a patient has a CRP>3 mg/L;
FEMALE=1 for a female patient and=0 for a male patient;
BMI=a patient's body mass index in units of kg/m², where kg is the body weight in kilograms and m² is the patient's height in meters squared.

19. The system of claim 15, wherein said multivariate function of said program product is:
% probability=1/(1+exp(−(0.13427+(0.81917*FEMALE)+(0.01489*AGE)−(0.00571*FEMALE*AGE)+0.21976*CHD+(0.10436*BMI)−(1.49181*alb)+(0.43003*LOGTRIG))))
where
% probability=the probability that a patient has a CRP>3 mg/L;
FEMALE=1 for a female patient and=0 for a male patient;
BMI=a patient's body mass index in units of kg/m², where kg is the body weight in kilograms and m² is the patient's height in meters squared;
AGE=age in years;
CHD=a doctor's diagnosis of prior coronary heart disease=1, else=0;
ALB=serum albumin in g/L; and
LOGTRIG=natural logarithm of serum triglycerides in mg/dL.
